(19) United States
(12) Patent Application Publication
(10) Pub. No.: US 2024/0303226 A1
(43) Pub. Date: Sep. 12, 2024

(54) SCALABLE DATA STRUCTURE BASED ON TRANSLATED EVENTS

(71) Applicant: Otsuka Pharmaceutical Development & Commercialization, Inc., Rockville, MD (US)

(72) Inventors: Osman Serdar TURKOGLU, Princeton, NJ (US); Arun JAIN, Monroe, NJ (US)

(73) Assignee: Otsuka Pharmaceutical Development & Commercialization, Inc., Rockville, MD (US)

(21) Appl. No.: 18/601,828

(22) Filed: Mar. 11, 2024

Related U.S. Application Data
(60) Provisional application No. 63/451,348, filed on Mar. 10, 2023.

(51) Int. Cl. G06F 16/21; G06F 16/28; G16H 20/10

(52) U.S. Cl. CPC G06F 16/211; G06F 16/287; G16H 20/10

(57) ABSTRACT

The disclosure relates to systems and methods of translating event data to generate a scalable data structure to identify or predict an event of interest such as a clinical diagnosis. The scalable data structure is expandable to accommodate various types of event data each having different types of timing indications on a timeline. The system may translate the event data in a way that event data can inherit event data values from other event data in a single time series of events. The scalable data structure may be used to generate unified visualizations of all translated events as well as for forecasting and predicting events of interest. The scalable data structure may be implemented in various contexts such as for clinical diagnostics in which clinical trial data or medical health data from various sources are translated to generate the scalable data structure.

### CROSS-REFERENCE TO RELATED APPLICATION

This application claims the benefit under 35 U.S.C. § 119(e) of U.S. Provisional Application No. 63/451,348, filed Mar. 10, 2023, the contents of which are incorporated by reference in its entirety.

### BACKGROUND

Data may be stored in various ways, such as using structured or unstructured data. Structured data refers to data that is able to be categorized or otherwise conforms to a data model. For example, a table having set rows and columns is structured data that conforms to the predefined columns. In many instances, storing data in a structured way makes the data easy to retrieve but harder to configure flexibly configure. In the table example, looking up a particular value in the columns is easy, but adding a new type of value that doesn't conform to the columns may not be possible. Storing data in an unstructured way may be more flexible, but harder to retrieve.

Datasets that may be individually structured but collectively does not follow a particular data model may be difficult to store in a structured way. Thus, what is needed is to be able to represent multiple datasets each being individually structured together but not following the same structure while leveraging easy-to-retrieve qualities of structured data for the multiple datasets. These and other issues exist when storing and retrieving the stored data.

### SUMMARY

The disclosure relates to systems and methods of translating event data to generate a scalable data structure. Event data may be received from various data sources. While each data source may provide respective event data that may be structured, collectively, the event data from multiple event sources may not be structured. One example of individual structured data includes timing data for an event. To illustrate, one data source may provide event data having time information represented as a date range in which event data values pertain to the entire date range. Another data source may provide event data having time information represented as a single date in which event data values pertain to only to the single date. Furthermore, one data source may provide certain types of data such as reported conditions or symptoms, while another data source may provide different types of data such as test results. These event data, while individually able to be structured, collectively may not be structured.

A system may address these and other issues by translating the event data to generate a scalable data structure. The scalable data structure is expandable to accommodate various types of event data each having different types of timing indications on a timeline. For example, the scalable data structure may include expandable columns based on different event data. The system may translate the event data in a way that event data can inherit event data values from other event data in a single time series of events. The scalable data structure may be used to generate unified visualizations of all translated events as well as for forecasting and predicting events of interest. The scalable data structure may be implemented in various contexts such as for clinical diagnostics in which clinical trial data or medical health data from various sources are translated to generate the scalable data structure. The scalable data structure may be implemented in other contexts in which data from different sources are not otherwise able to be structured together, such as in computer networks in which data sources provide event data in different ways.

In some implementations, the disclosure relates to a system including: a processor programmed to: access first event data including a first event data value and a corresponding first date range for which the first event data value pertains; access second event data including a second event data value and a corresponding second date for which the second event data value pertains; generate a structured schema for a scalable data structure in which a number of columns is based on a number of distinct values derived from the first event data and the second event data; translate the first event data and the second event data into a time series of events in which: (a) the first event data value in the first event data is associated with the single date in second event data and (b) the second event data value in the second event data is associated the first date range in the first event data; populate the scalable data structure based on the structured schema and the translated time series of events, wherein a number of a plurality of rows is based on a start date in the first date range, an end date in the first date range, and the second single date in the second event data and wherein each row from among the plurality of rows has a plurality of columns each corresponding to the distinct values derived from the first event data and the second event data; and generate, for display, a visualization based on the populated scalable data structure.

In some implementations, to generate the structured schema, the processor is further programmed to: parse the first event data value from the first event data; and generate a first column in the structured schema, the first column having a first column name based on the first event data value.

In some implementations, to generate the structured schema, the processor is further programmed to: parse the second event data value from the second event data; and generate a second column in the structured schema, the second column having a second column name based on the second event data value.

In some implementations, to translate the first event data, the processor is further programmed to: determine whether the single date in the second event data is equal to the start date of the first date range; generate a binarized value based on the second event data value and the determination of whether the single date in the second event data is equal to the start date of the first date range; store the binarized value as a column value of a column for a row corresponding to the start date.

In some implementations, to translate the second event data, the processor is further programmed to: determine whether the single date in the second event data is equal to the end date of the first date range; generate a second binarized value based on the second event data value and the determination of whether the single date in the second event data is equal to the end date of the first date range; store the second binarized value as a second column value of a second column for a second row corresponding to the end date.

In some implementations, to translate the second event data, the processor is further programmed to: determine whether the single date in the second event data is within the first date range; generate a binarized value based on the first event data value and the determination of whether the single date in the second event data is within the first date range; store the binarized value as a column value of a column for a row corresponding to the single date.

In some implementations, the disclosure relates to a system in which the first event data value pertains to a symptom that was reported during the first date range.

In some implementations, the disclosure relates to a system in which the second event data value pertains to a test result that was obtained at the single date.

In some implementations, to generate the visualization, the processor is further programmed to: generate a timeline based on rows in the scalable data structure; for each row in the scalable data structure: for each column in the scalable data structure, determine whether a column value for the column represents an event of interest and generate an event marker along the timeline corresponding to the row depending on whether the column value for the column represents an event of interest.

In some implementations, the disclosure relates to a method, including: accessing, by a processor, first event data including a first event data value and a corresponding first date range for which the first event data value pertains; accessing, by the processor, second event data including a second event data value and a corresponding second date for which the second event data value pertains; generating, by the processor, a structured schema for a scalable data structure in which a number of columns is based on a number of distinct values derived from the first event data and the second event data; translating, by the processor, the first event data and the second event data into a time series of events in which: (a) the first event data value in the first event data is associated with the single date in second event data and (b) the second event data value in the second event data is associated the first date range in the first event data; populating, by the processor, the scalable data structure based on the structured schema and the translated time series of events, wherein a number of a plurality of rows is based on a start date in the first date range, an end date in the first date range, and the second single date in the second event data and wherein each row from among the plurality of rows has a plurality of columns each corresponding to the distinct values derived from the first event data and the second event data; and generating, by the processor, for display, a visualization based on the populated scalable data structure.

In some implementations, generating the structured schema includes: parsing the first event data value from the first event data; and generating a first column in the structured schema, the first column having a first column name based on the first event data value.

In some implementations, generating the structured schema includes: parsing the second event data value from the second event data; and generating a second column in the structured schema, the second column having a second column name based on the second event data value.

In some implementations, translating the first event data includes: determining whether the single date in the second event data is equal to the start date of the first date range; generating a binarized value based on the second event data value and the determination of whether the single date in the second event data is equal to the start date of the first date range; and storing the binarized value as a column value of a column for a row corresponding to the start date.

In some implementations, translating the second event data includes: determining whether the single date in the second event data is equal to the end date of the first date range; generating a second binarized value based on the second event data value and the determination of whether the single date in the second event data is equal to the end date of the first date range; and storing the second binarized value as a second column value of a second column for a second row corresponding to the end date.

In some implementations, translating the second event data includes: determining whether the single date in the second event data is within the first date range; generating a binarized value based on the first event data value and the determination of whether the single date in the second event data is within the first date range; and storing the binarized value as a column value of a column for a row corresponding to the single date.

In some implementations, the disclosure relates to a method in which the first event data value pertains to a symptom that was reported during the first date range.

In some implementations, the disclosure relates to a method in which the second event data value pertains to a test result that was obtained at the single date.

In some implementations, the method further includes: generating a timeline based on rows in the scalable data structure; for each row in the scalable data structure: for each column in the scalable data structure, determining whether a column value for the column represents an event of interest and generating an event marker along the timeline corresponding to the row depending on whether the column value for the column represents an event of interest.

In some implementations, the disclosure relates to a non-transitory computer readable medium storing instructions that, when executed by a processor, programs the processor to: access first event data including a first event data value and a corresponding first date range for which the first event data value pertains, the first date range having a start date and an end date; access second event data including a second event data value and a corresponding second date for which the second event data value pertains; determine whether the single date in the second event data is equal to the start date of the first date range; generate a binarized value based on the second event data value and the determination of whether the single date in the second event data is equal to the start date of the first date range; store the binarized value in association with the start date; determine whether the single date in the second event data is equal to the end date of the first date range; generate a second binarized value for the second event data value based on the determination of whether the single date in the second event data is equal to the end date of the first date range; and store the second binarized value in association with the end date.

In some implementations, the non-transitory computer readable medium storing instructions, wherein the instructions, when executed, further programs the processor to: determine whether the single date in the second event data is within the first date range; generate a third binarized value based on the first event data value and the determination of whether the single date in the second event data is within the first date range; store the third binarized value in association with the single date.

### BRIEF DESCRIPTION OF THE DRAWINGS

Features of the present disclosure may be illustrated by way of example and not limited in the following Figure(s), in which like numerals indicate like elements, in which:

FIG. 1 illustrates an example of a system for generating a scalable data structure by translating event data having variable time scales and values into a unified time series;

FIG. 2 illustrates an example of translating time series data for configuring and populating the scalable data structure;

FIG. 3A illustrates an example of instantiating the scalable data structure;

FIG. 3B illustrates an example of populating the scalable data structure;

FIG. 3C illustrates an example of populating and then sorting the scalable data structure;

FIG. 4 illustrates an example of dynamically scaling the scalable data structure to accommodate newly received types of events;

FIG. 5 illustrates an example of a visualization for displaying data from the scalable data structure for analysis;

FIG. 6 illustrates an example of a method of configuring and populating a scalable data structure based on event data that includes independent events;

FIG. 7 illustrates an example of a method of configuring, populating and using the scalable data structure to predict a physiological state of a subject;

FIG. 8 illustrates an example of a method of translating event data;

FIG. 9 illustrates an example of a computing system implemented by one or more of the features illustrated in FIG. 1; and

FIG. 10 illustrates an example of a method of treatment.

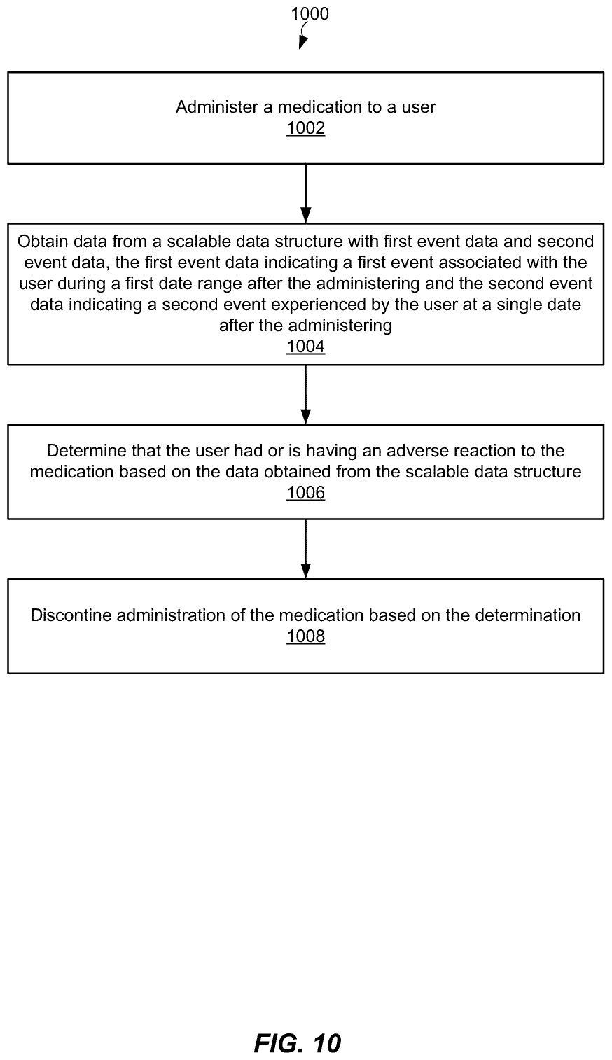

### DETAILED DESCRIPTION

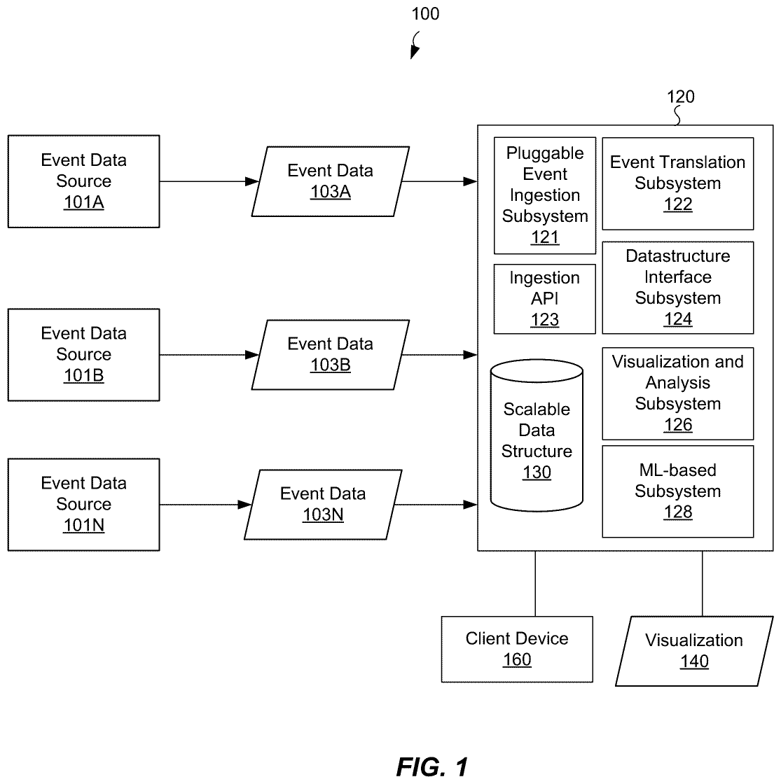

FIG. 1 illustrates an example of a system 100 for generating a scalable data structure 130 by translating event data 103 having variable time scales and values into a unified time series. System 100 may include a plurality of event data sources 101 (illustrated as event data sources 101A-N), a computer system 120, a scalable data structure 130, a client device 160, and/or other features. The various components of the system 100 may be connected to one another via one or more networks.

One or more of the networks may be a communications network including one or more Internet Service Providers (ISPs). Each ISP may be operable to provide Internet services, telephonic services, and the like, to one or more devices, such as the computer system 120 and computing device 140. In some examples, one or more of the networks may facilitate communications via one or more communication protocols, such as those mentioned above (for example, TCP/IP, HTTP, WebRTC, SIP, WAP, Wi-Fi (for example, 802.11 protocol), Bluetooth, radio frequency systems (for example, 900 MHz, 1.4 GHz, and 5.6 GHz communication systems), cellular networks (for example, GSM, AMPS, GPRS, CDMA, EV-DO, EDGE, 3GSM, DECT, IS 136/TDMA, iDen, LTE or any other suitable cellular network protocol), infrared, BitTorrent, FTP, RTP, RTSP, SSH, and/or VOIP.

The computer system 120 may be implemented as a cloud-based system and/or an on-premises system that access event data 103 from one or more event data sources. An event data source 101 may be a device or service that provides event data 103. The type of event data 103 provided by an event data source 101 will depend on the context in which the system 100 is implemented. For example, in computer network contexts, the event data 103 may include network event data such as a logon attempt, a service or other network request, and/or other network-related data. In clinical health contexts, the event data 103 may include health symptoms of a subject, clinical test results of the subject, and/or other clinical health related data. Other contexts may be associated with other types of event data. Regardless of the particular context, each event data 103 may have data record formats and values that differ from one another.

The computer system 120 may translate the event data 103 and generate a scalable data structure 130. For example, the computer system 120 may include an event translation subsystem 122, a data structure interface subsystem 124, a visualization subsystem 126, a machine learning (ML)-based subsystem 128, and/or other features. Some or all of the subsystems and/or features may be implemented in hardware or software that programs hardware.

### Parsing and Encoding Event Data for Translation

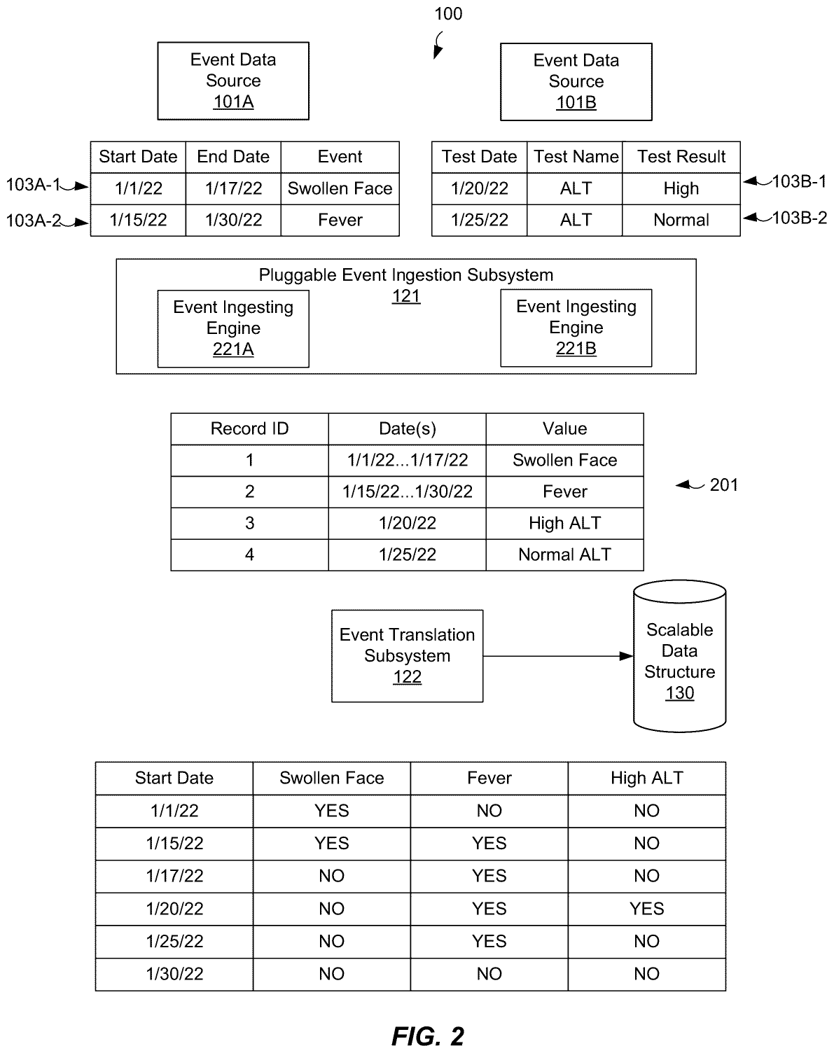

The pluggable event ingestion subsystem 121 may ingest event data 103 based on the format and type of data encoded by the event data to translate the event data 103. The term “translating” and similar terms may refer to placing first event data of a first event having at least a first associated date at which the first event occur in a time series of events along with other event data having at least a second associated date and inheriting values of the other event data for the first associated date, and vice versa. Referring to FIG. 2, for example, the pluggable event ingestion subsystem 121 may include a plurality of event ingesting engines 221 (illustrated as event ingesting engines 221A and 221B). In some examples, each event ingesting engine 221 may be specifically configured to ingest event data 103 from a corresponding event data source 101. For example, an event ingesting engine 221A may ingest data records 103A-1 and 103A-2 from event data source 101A. Similarly, an event ingesting engine 221B may ingest data records 103B-1 and 103B-2 from event data source 101B. In this way, the pluggable event ingestion subsystem 121 may scale to access and transform data from a wide range of event data sources 101 and/or to deprecate unneeded or no longer used event data sources, reducing computational overhead of unneeded ingesting engines.

In some examples, the pluggable event ingestion subsystem 121 may ingest event data 103 based on one or more Application Programming Interface (API) calls to an ingestion API 123. The API calls exposed by the ingestion API 123 may standardize the way in which event data is ingested to the system, thereby enabling consistent data inputs. For example, the one or more API calls may include an add event API call. The add event API call may include various input parameters such as a date input parameter, an event name input parameter, an event value parameter, a binarization threshold parameter, and/or other parameters for adding an event for translation into the scalable data structure 130. Other types of API calls may be exposed by the ingestion API 123 as well.

An event ingesting engine 221 may parse event data 103 and execute the add event API call to provide the information parsed from event data 103. The event ingesting engine 221 may include various input parameters such as the date input parameter, an event name input parameter, an event value parameter, a binarization threshold parameter.

The date input parameter may specify a date value. The date value may be a single date, a range of dates separated by a range separate, a list of dates separated by a list separator, and/or other type of date value. The ingestion API 123 may enforce requirements of the date value, such as by requiring a certain date format (such as MM/DD/YY), requiring specific types of date range or list separators, and/or other requirements for the value of the date parameter.

When an event ingesting engine 221 invokes the date input call, the ingestion API 123 may inspect the input parameters and store the values in a memory buffer for ingestion along with the remaining portions of the event record.

An event ingesting engine 221 may execute the event name input call to provide event names parsed from event data 103. The event name input call may include input parameters such as an event name parameter, an event value parameter, an event date parameter, a binarization threshold value parameter, and/or other input parameters.

The event name input parameter may specify an event name. The event name may be a variable character that includes text, numbers, and/or other characters. The event name may generally be free-form to be able to capture different types of events, such as “Fever,” “Swollen Face,” “Test Result” and so forth.

The event value input parameter may specify an event value. The event value may specify a value for the event name. For example, the event name “Event” input at the event name input call may have a corresponding value of “Fever.” The event name “Test Result” input at the event name input call may have a corresponding value of “High ALT.”

The binarization threshold input parameter may specify a binarization threshold value such that may be compared to a quantitative value for the event value specified in the event value input call. The binarization threshold input call may therefore be optionally called for certain types of events such as “Test Results.” In these examples, if the event name “Test Result” has a value of value of 0.8 and the binarization threshold value is 0.7, then the binarization may result in a value of “HIGH” or “1” or other binary indication.

It should be noted that instead of the binarization threshold input call, the ingestion API 123 may instead receive a binarized value from the calling event ingesting engine 221. In this example, the calling event ingesting engine 221 may binarize the event data. In other words, the ingestion API 123 in these examples may simply receive a binarized result for a given event name without having to compare a binarization threshold value to the input event value.

As illustrated, event data 103A-1 and 103A-2 from event data source 1A may have a date range and an event associated with the date range. The date range has a start date and an end date. In this example, the illustrated event is a health symptom of the subject and the date range indicates a duration in which the subject experienced the health symptom. Event data 103B-1 and 103B-2 from event data source 1B may have a single date, a test name, and a result of the test identified by the test name. The examples shown in FIG. 2 relate to a clinical health context for illustrative purposes. Other contexts may be used as well or instead.

Each event ingesting engine 221 may ingest event data and encode data for translation based on rules-based logic. In these examples, the event ingesting engine 221 may access one or more rules that are specifically executed to ingest specific event data in specific ways. For example, an event ingesting engine 221A may ingest data from a data source 101A that provides clinical presentation of symptoms. The event ingesting engine 221A may access rules that specify that an event value such as “Swollen Face” should be parsed along with one or more dates. The rules may further specify that this event data should be translated as a binary value for the one or more dates to indicate that the condition “Swollen Face” was reported on the one or more dates (whether a range of dates and/or one or more single dates). For example, an event ingesting engine 221A may parse the “Swollen Face” event value and the date range 1/1//22 to 1/17/22 from the event data 103A-1 and access one or more rules that specify that this event data be translated to a record in the scalable data structure 130 that indicates a binary value of “YES” for the date range 1/1//22 to 1/17/22 to indicate that “Swollen Face” was present during this time.

Other rules may be similarly applied by other event ingesting engines 221. For example, an event ingesting engine 221B may ingest data from a data source 101B that provides test results. Test results may vary in the type and format of the results. As such, different rules may be applied to different types of test results. The event ingesting engine 221B may access rules that specify that an event value such as “High” or “Normal” should be parsed along with one or more dates. The rules may further specify that this event data should be translated as a binary value for the one or more dates to indicate that a Test Name such as “ALT” should be combined with the event value to form “ALT High” or “ALT Normal” on the one or more dates (whether a range of dates and/or one or more single dates). For example, an event ingesting engine 221B may parse the Test Name “ALT”, the event value “High” and test result date of 1/20/22 from the event data 103B-1 and access one or more rules that specify that this event data be translated to a record in the scalable data structure 130 that indicates a binary value of “YES” for the date 1/20/22 to indicate that the “ALT” test result was “High” (or “Normal” for event data 103B-2). It should be noted that, in some examples, the rules may specify binarization as well or instead. In these examples, the binarization rule may specify that an “ALT High” result be binarized to “YES” or “1” (or other binary indication) to indicate a high value for the ALT test. In other examples, the event translation subsystem 122 may binarize the event values, such as based on the binarization threshold parameter.

It should be further noted that instead of “High” or “Normal” the Test Result value parsed from the event data 103 may include a quantitative value, such as a numeric value. In these instances, the event ingesting engine 221 may use the binarization threshold parameter to binarize the quantitative value. In some examples, the binarization threshold parameter may specify that the term “High” (or “Low” depending on the nature of the test) means that the result should be binarized to indicate a high (or low) value while all other terms may be binarized to indicate a normal value. It should be noted that the rules may be tailored to specific types of tests or event data 103. Thus, the pluggable event ingestion subsystem 121 may be scaled to ingest various types of data as needed (and remove ingestion processing for unneeded or deprecated data sources).

Whether rules-based or ML-based, the pluggable event ingestion subsystem 121 may generate a temporary lookup table 201 based on the ingested and encoded event data. The pluggable event ingestion subsystem 121 may store the temporary lookup table 201 in a memory cache, such as a memory buffer that may be removed when the scalable data structure 130 is instantiated and populated. The temporary lookup table 201 may store a record ID that identifies a record from the event data 103 that was parsed. The record ID may be generated by the pluggable event ingestion subsystem 121 for each event data 103 that is parsed. This is to ensure that the same date from different events can be distinguished from one another. The temporary lookup table 201 may include different ways to store dates encoded in event data 103. In the illustrated example, a date column in the temporary lookup table 201 may store date ranges as a start_date and an end_date separated by a range separator such as ellipses (“ . . . ”). Other range separators may be used. Dates without range separators are single dates. It should be noted that multiple single dates relating to the same event may be listed separately or separated by a list separator such as a comma (“,”). Other list separators may be used as well. It should be further noted that the date column in the temporary lookup table 201 may a combination of date ranges, date lists, single dates, and/or other ways in which to encode dates, so long as one or more dates from event data 103 is maintained for a given record ID. The temporary lookup table 201 may further include a value column in which a value from the event data 103 is stored. This stored value may have been encoded by the specific event ingesting engine 221.

### Scalable Data Structure Based on Event Data

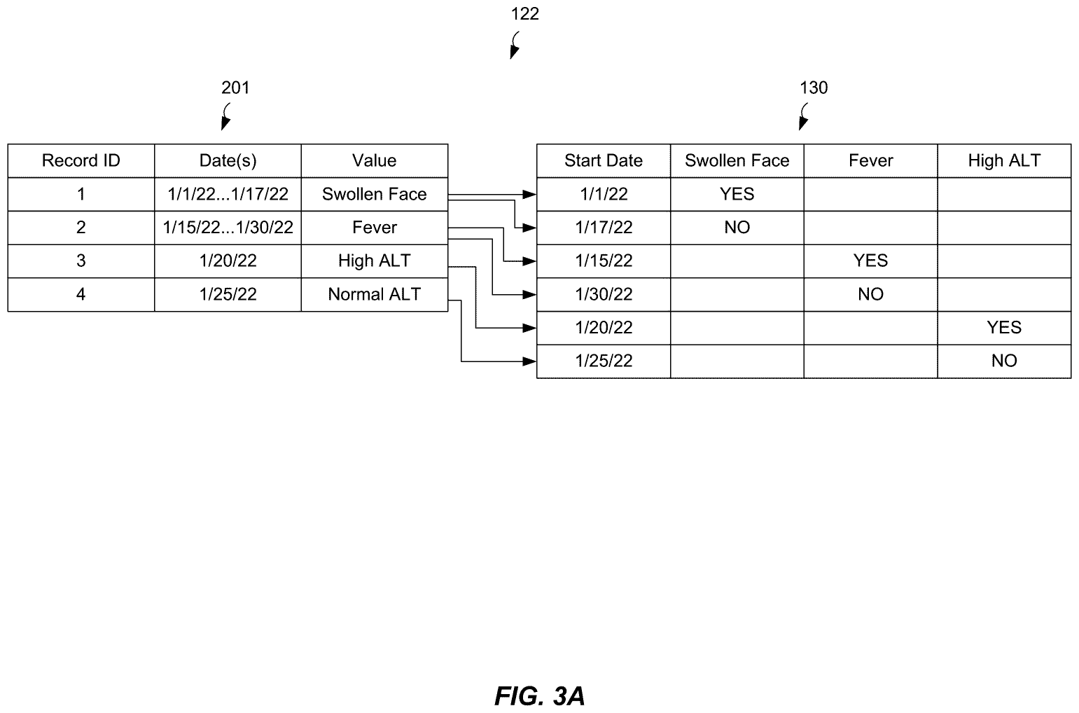

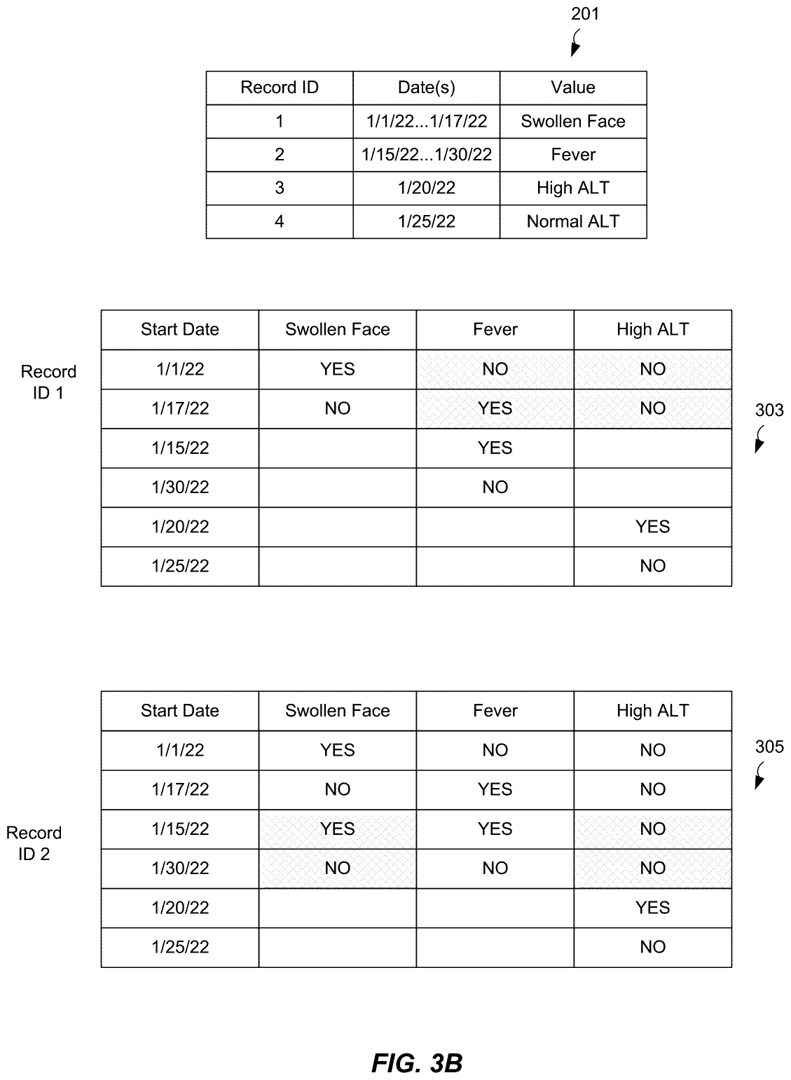

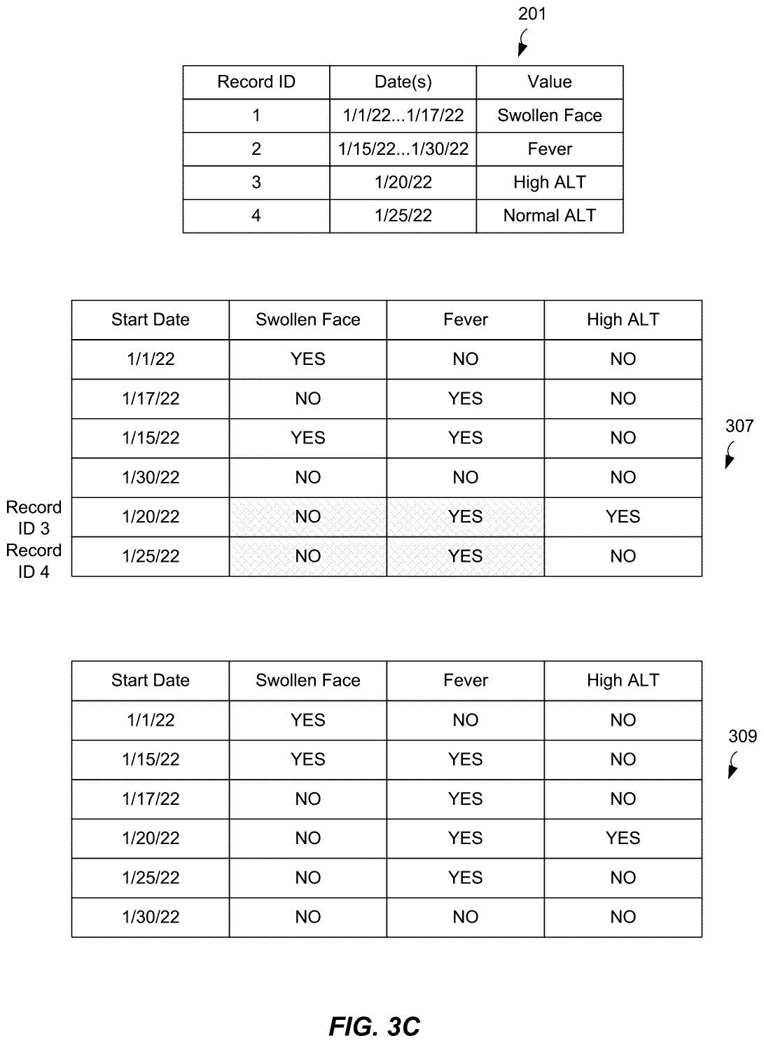

The event translation subsystem 122 may generate a scalable data structure 130 based on the event data 103A-N parsed by the pluggable event ingestion subsystem 121. It should be noted that each row in the scalable data structure 130 may also include an identifier for a subject to which the event data pertains. This identifier is not shown for clarity, but may be used to store data for multiple subjects. To illustrate, reference will be made to FIG. 3A, which illustrates an example of instantiating the scalable data structure 130. The event translation subsystem 122 may generate the scalable data structure 130 based on a structured schema that is dynamically adjusted based on the event data 103. The structured schema may refer to a number of rows, a number of columns, and type of columns of the of the scalable data structure 130. A type of column may include a column name and a column value. The event translation subsystem 122 may translate the event data 103 into the structured schema of the scalable data structure 130. For example, event translation subsystem 122 may generate, for each record ID, a row in the scalable data structure 130 for each date (including for each start date in a range and each end date in the range) in the temporary lookup table 201 and a column based on each distinct value column in the temporary lookup table 201.

The event translation subsystem 122 may access the temporary lookup table 201 generated by the pluggable event ingestion subsystem 121. The event translation subsystem 122 may identify the dates in the date column for each record ID 1-4. These dates were parsed by the pluggable event ingestion subsystem 121 and populated in the date column. For example, record ID 1 and record ID 2 illustrated in FIG. 3A has two date ranges, each with a start_date and an end_date, and record ID 3 and record ID 4 each have a single date. In this example, the event translation subsystem 122 may generate six rows corresponding to the dates (four start and end dates and two single dates) in the temporary lookup table 201.

The event translation subsystem 122 may generate a column based on each value in the temporary lookup table 201, which was parsed from the data records by the pluggable event ingestion subsystem 121. At least some of the columns may have a binarized value. For example, the event translation subsystem 122 may generate columns “swollen face”, “high fever”, and “high ALT” based on the distinct values parsed from event data 103 and stored in the temporary lookup table 201. Thus, the event translation subsystem 122 may generate columns in the scalable data structure 130 based on distinct values of event data 103.

The event translation subsystem 122 may populate the scalable data structure 130 based on the temporary lookup table 201. For example, the event translation subsystem 122 may, for each record ID in the temporary lookup table 201, populate its corresponding values. For example, for record ID 1, the value “Swollen Face” started on start_date 1/1/22 and ended on end_date 1/17/22. Accordingly, the event translation subsystem 122 may populate the corresponding start_date of 1/1/22 with a column value of “YES” for the “Swollen Face” column in the scalable data structure 130 and the corresponding end_date of 1/17/22 with a column value of “NO” for the “Swollen Face” column in the scalable data structure 130. Other binarized values such as 1 to indicate “YES” and 0 to indicate “NO” may be used instead.

The event translation subsystem 122 may continue populating the scalable data structure 130 for each row and corresponding record ID. For example, for record ID 2, the event translation subsystem 122 may populate the corresponding start_date of 1/15/22 with a column value of “YES” for the “Fever” column in the scalable data structure 130 and the corresponding end_date of 1/30/22 with a column value of “NO” for the “Fever” column in the scalable data structure 130. For record ID 3, the event translation subsystem 122 may populate the corresponding start_date of 1/20/22 with a column value of “YES” for the “High ALT” column in the scalable data structure 130. For record ID 4, the event translation subsystem 122 may populate the corresponding start_date of 1/25/22 with a column value of “NO” for the “High ALT” column in the scalable data structure 130.

The event translation subsystem 122 may populate remaining unfilled columns by translating data from other event data stored in the temporary lookup table 201. Generally, the event translation subsystem 122 may, for each record ID, determine whether a date for that record ID is within or matches the dates of other record IDs. If so, then the event translation subsystem 122 will binarize the value of the matching record ID's value column to the current record ID's column value. To illustrate, reference will be made to FIGS. 3B and 3C.

Referring first to FIG. 3B, the event translation subsystem 122 may populate missing columns of one or more rows corresponding to each record ID. For example, as schematically illustrated at 303, the event translation subsystem 122 may populate (“Fever” and “High ALT”) of rows pertaining to record ID 1. In this case, two rows based on dates 1/1/22 and 1/17/22 were generated for record ID 1. The event translation subsystem 122 may populate missing columns of each of this record ID's rows. For example, the event translation subsystem 122 may lookup the date 1/1/22 in the temporary lookup table 201 to determine whether that date matches other record ID's dates or is within a range of other record ID's dates. In particular, the event translation subsystem 122 may determine that 1/1/22 does not fall within the range of the dates of record ID 2 in the temporary lookup table 201. As such, the resulting column entry for this row's “FEVER” column is binarized to be “NO.” Similarly, the event translation subsystem 122 may determine 1/1/22 does not match the date of record ID 3. As such, the resulting column entry for this row's “High ALT” column is binarized to be “NO.” The event translation subsystem 122 may continue through other record IDs such as record ID 4 and determine 1/1/22 does not match the date of record ID 4. As such, the resulting column entry for this row's “High ALT” column is binarized to be “NO.”

Repeating this process for the second row (1/17/22) generated for record ID 1, the event translation subsystem 122 may determine that 1/17/22 falls within the range of the dates of record ID 2 in the temporary lookup table 201. As such, the resulting column entry for this row's “FEVER” column is binarized to be “YES.” The event translation subsystem 122 may also determine that 1/17/22 does not match the date of record ID 3. As such, the resulting column entry for this row's “High ALT” column is binarized to be “NO.” The event translation subsystem 122 may continue through other record IDs such as record ID 4 and determine 1/17/22 does not match the date of record ID 4. As such, the resulting column entry for this row's “High ALT” column is binarized to be “NO.”

At 305, the event translation subsystem 122 may similarly populate missing column values for the rows of record ID 2. Two rows based on dates 1/15/22 and 1/30/22 were generated for record ID 2. The event translation subsystem 122 may populate missing columns of each of this record ID's rows. For example, the event translation subsystem 122 may lookup the date 1/15/22 in the temporary lookup table 201 to determine whether that date matches other record ID's dates or is within a range of other record ID's dates. In particular, the event translation subsystem 122 may determine that 1/15/22 falls within the range of the dates of record ID 1 in the temporary lookup table 201. As such, the resulting column entry for this row's “Swollen Face” column is binarized to be “YES.” The event translation subsystem 122 may determine that 1/15/22 does not match the date of record ID 3. As such, the resulting column entry for this row's “High ALT” column is binarized to be “NO.” The event translation subsystem 122 may continue through other record IDs such as record ID 4 and determine that 1/15/22 does not match the date of record ID 4. As such, the resulting column entry for this row's “High ALT” column is binarized to be “NO.”

Repeating this process for the second row (1/30/22) generated for record ID 2, the event translation subsystem 122 may determine that 1/30/22 does not fall within the range of the dates of record ID 1 in the temporary lookup table 201. As such, the resulting column entry for this row's “Swollen Face” column is binarized to be “NO.” The event translation subsystem 122 may determine that 1/30/22 does not match the date of record ID 3. As such, the resulting column entry for this row's “High ALT” column is binarized to be “NO.” The event translation subsystem 122 may continue through other record IDs such as record ID 4 and determine that 1/30/22 does not match the date of record ID 4. As such, the resulting column entry for this row's “High ALT” column is binarized to be “NO.”

Referring now to FIG. 3C, at 307, the event translation subsystem 122 may similarly populate missing column values for the row of record IDs 3 and 4. Record IDs 3 and 4 each include a single row based on respective dates 1/20/22 and 1/25/22. The event translation subsystem 122 may populate missing columns of each of these record ID's row. For example, the event translation subsystem 122 may lookup the date 1/20/22 in the temporary lookup table 201 to determine whether that date matches other record ID's dates or is within a range of other record ID's dates. In particular, the event translation subsystem 122 may determine that 1/20/22 does not fall within the range of the dates of record ID 1 in the temporary lookup table 201. As such, the resulting column entry for this row's “Swollen Face” column is binarized to be “NO.” The event translation subsystem 122 may determine that 1/20/22 falls within the range of dates in record ID 2. As such, the resulting column entry for this row's “Fever” column is binarized to be “YES.”

Repeating this process for the date from record ID 2, the event translation subsystem 122 may determine that 1/25/22 does not fall within the range of the dates of record ID 1 in the temporary lookup table 201. As such, the resulting column entry for this row's “Swollen Face” column is binarized to be “NO.” Similarly, the event translation subsystem 122 may determine that 1/25/22 falls within the date range of record ID 2. As such, the resulting column entry for this row's “Fever” column is binarized to be “YES.”

At 309, the event translation subsystem 122 may sort the scalable data structure 130 based on the date. Thus, the scalable data structure 130 may represent a time series of events merged from event data having multiple date ranges, points in time, and/or other time indications. The scalable data structure 130 may be interrogated by the data structure interface subsystem 124.

Updating and Scaling the Scalable Data Structure with New Fields

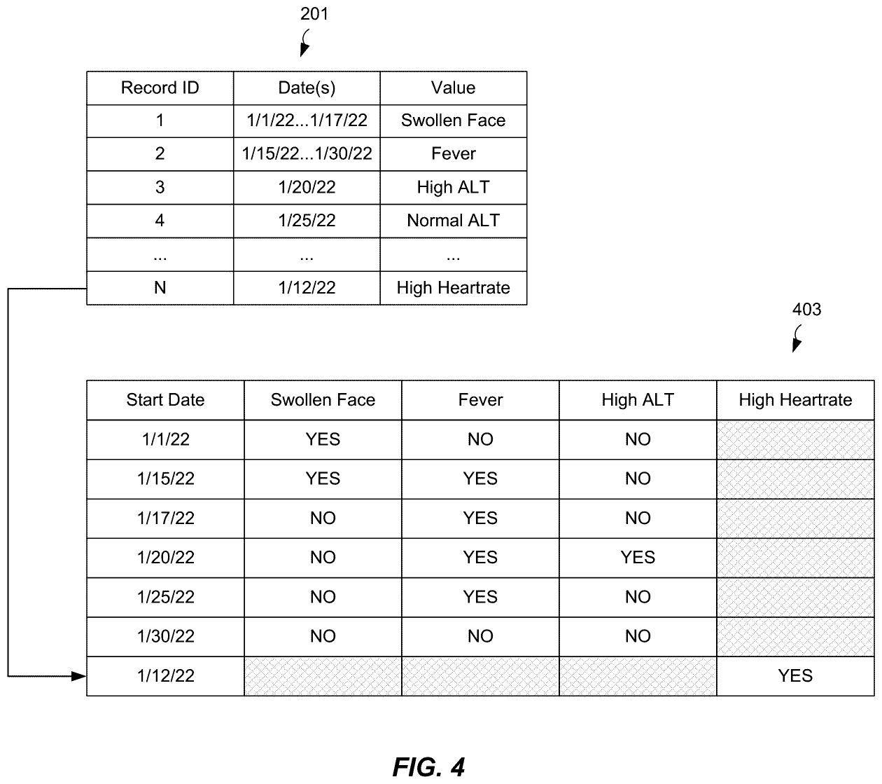

Once generated and populated, the scalable data structure 130 may be updated in a manner similar to that described with respect to FIGS. 3A-3C as incoming event data 103 is received. In some examples, event data 103 received after the scalable data structure 130 has been generated and populated may include data fields not previously seen therefore not in the scalable data structure 130. To illustrate, reference will be made to FIG. 4, which illustrates an example of dynamically scaling the scalable data structure 130 to accommodate newly received types of events. Referring to FIG. 4, the temporary lookup table 201 may be updated based on incoming event data 103, including a new type of event data for heartrate. For example, the pluggable event ingestion subsystem 121 (such as by a specific event ingesting engine 221 that was added to handle the new type of event data) may ingest the new type of event data and generate record ID (N). The date value indicates that the subject had a high heartrate reported on 1/12/22, although any other date and type of value may have been received, translated, and recorded in the temporary lookup table 201.

The event translation subsystem 122 may periodically monitor the temporary lookup table 201 to update the scalable data structure 130. The event translation subsystem 122 may recognize that a new value (“High Heartrate”) exists in the temporary lookup table 201 and modify the scalable data structure 130. For example, as illustrated at 403, the event translation subsystem 122 may generate a new column (“High Heartrate”) for the new value, generate a new row for the associated date (“1/12/22”) and enter a binarized value for the new column of the new row. The event translation subsystem 122 may then populate existing columns (“Swollen Face,” “Fever,” and “High ALT”) for the new row similar to the manner described at FIGS. 3A-3C. Similarly, the event translation subsystem 122 may back-populate the new column “High Heartrate” for the existing rows also in a manner similar to described at FIGS. 3A-3C. In this way, the event translation subsystem 122 may dynamically scale the scalable data structure 130 based on new types of events and corresponding new types of values of those events.

Once populated and/or dynamically scaled, the scalable data structure 130 may be used for further analysis. For example, the data structure interface subsystem 124 may execute requests such as database queries to access data from the scalable data structure 130. The requests may be used to identify relevant events in time that correlate with one another in order to make predictions on future events and/or perform post-action analyses to determine what caused a prior event.

Visualizing Translated Events from the Scalable Data Structure

The visualization subsystem 126 may generate a visualization 140 based on the scalable data structure 130. For example, the visualization subsystem 126 may access the scalable data structure 130 via the data structure interface subsystem 124 to obtain data for the visualization 140. Such access may be via direct queries to and/or through APIs exposed by the data structure interface subsystem 124. The visualization 140 may be transmitted for display at a client device 160. For example, the visualization 140 may include or be part of a user interface displayed at the client device 160.

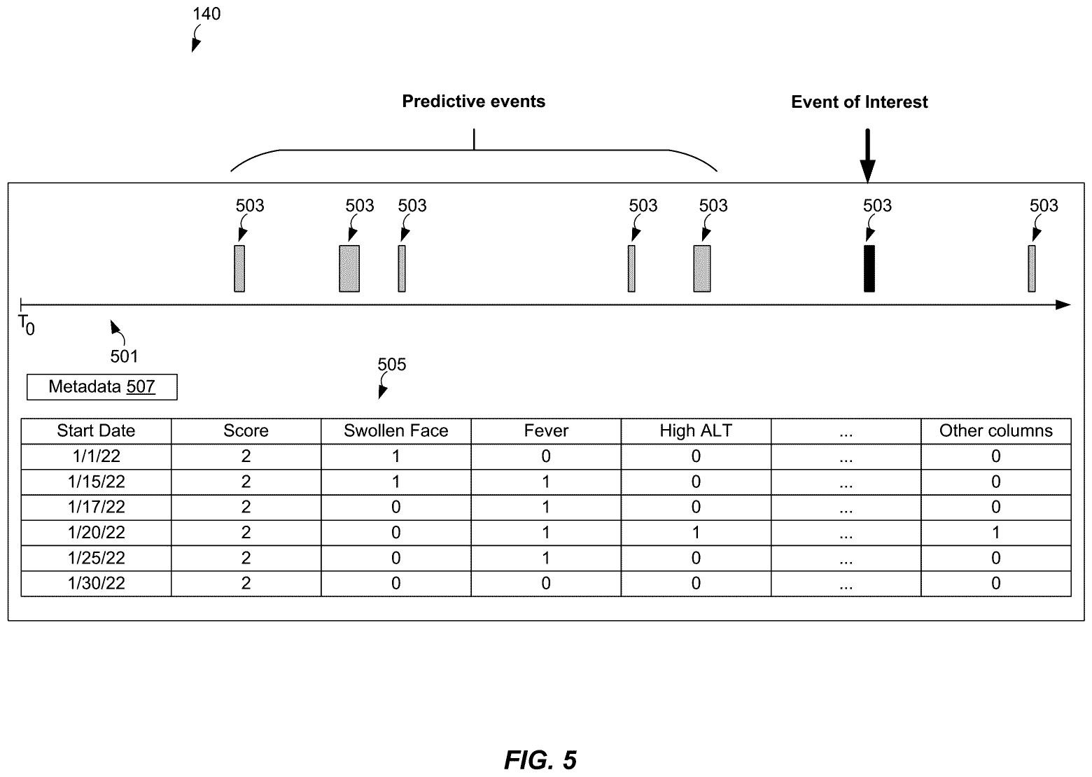

FIG. 5 illustrates an example of the visualization 140 for displaying data from the scalable data structure 130 for analysis. FIG. 5 will be described in a clinical context for illustration. As previously noted, the disclosure herein may be applied in other contexts. One context that may be used is for clinical observations of clinical trial subjects or patients undergoing or seeking therapy to monitor effects of drugs. The visualization 140 may include event markers 503 along a timeline 501 starting from T₀. T₀ is a start date after which events are to be observed and visualized. For example, T₀ may be an starting date input value to filter events to retrieve event data from the scalable data structure 130 that occur on or after the starting date input value. Although not illustrated, an ending date input value may be used to filer events from the scalable data structure 130 as well or instead. Chart 505 is a visual representation of the event data, which was accessed from the scalable data structure 130, from which the event markers 503 were generated. Metadata 507 is data relating to the event data such as a description of the context in which the event data relates. For example, metadata 507 may include a study identifier for identifying a clinical trial, a subject identifier (which may be anonymized) for identifying a subject in the clinical trial, and/or other data.

The event markers 503 may each represent an event value and the date associated with the event value. Each event marker 503 may be positioned along the timeline 501 according to its respective date. In some examples, each event marker 503 may be sized, shaped, colored, or otherwise distinguished from other event markers 503 to distinguish duration of the corresponding event, relative importance of the corresponding event, and/or other distinguishing properties of the underlying event data. Selection (such as clicking on or otherwise identifying) an event marker 503 may cause the event data underlying the event marker to be displayed. For example, such event data may be retained in a temporary memory location at the client device 160 and retrieved and displayed from the temporary memory location. Alternatively, the visualization 140 may cause a callback to the visualization subsystem 126, which may access the scalable data structure 130 to obtain the relevant event data.

In some examples, there may be an event of interest that is to be correlated with other events. For example, predictive events may be correlated with the event of interest. In some examples, correlation means that the predictive events are coincident (occur together) with the event of interest. By providing the event markers 503 along the timeline 501, the visualization 140 provides a spacing and frequency of events, as well as event type describes at the chart 505, that enables identification of the predictive events relative to the event of interest. The visualization 140 may therefore provide correlative and predictive capabilities to identify predictive events for events of interest.

To generate the visualization 140, the visualization subsystem 126 may receive inputs from a user, such as a clinician, clinical trial operator, and/or others involved in the observation or care of a subject such as a clinical trial participant, a patient undergoing or seeking therapy, and/or others whose health is being observed or treated. For example, the inputs may include a starting input date (illustrated as T₀), an ending input date, metadata that identifies events to be retrieved (such as a study identifier that identifies a clinical trial and its associated subjects and/or event data), types of event data to collect, and/or other inputs. In operation, the visualization subsystem 126 may generate the visualization 140 on-demand, such as through a request from a client device 160. Alternatively, or additionally, the visualization subsystem 126 may generate the visualization 140 periodically and push the visualization 140 to the client device 160 or otherwise make the visualization 140 available upon generation. In either case, the visualization subsystem 126 may generate the visualization 140 based on current translated event data, which may have been updated

The visualization 140 may facilitate event analysis such as root cause analysis in computer networks, or clinical diagnostics in medical decision support systems. A particular example in clinical diagnostics will be described in which the visualization 140 and, more generally, translated event data 103 into the scalable data structure 130 improves forecasting and root cause analysis. In many areas of clinical diagnostics, it may be difficult to identify events such as clinical symptoms or test results that correlate with disease states. This is because the body (whether human or other subject) is a complex biological and biochemical system. Simply put, given an observed disease state, it may be difficult to identify symptoms or test results that correlate with the observed disease state. By extension, it may be difficult to accurately predict that the disease state will occur in the subject even though clinical symptoms, test results, or other events known about the subject can be determined in real-time or through event data, such as historical medical or clinical trial records.

For example, Drug Reaction with Eosinophilia and Systemic Symptoms (DRESS) is a severe adverse reaction to a drug that may result in various conditions such as an extensive skin rash in association with visceral organ involvement, lymphadenopathy, eosinophilia, atypical lymphocytosis, and/or other observable or testable conditions. A subject may experience DRESS after being administered with the drug. Not all subjects will experience DRESS in reaction to the drug and it is important to promptly identify the onset of DRESS to discontinue administration of the drug. However, oftentimes it may be difficult to identify DRESS as it begins to occur or is occurring. This may result in prolonged administration of the drug and corresponding DRESS conditions. The difficulty may be especially acute and important in a clinical study setting in which multiple subjects are given the drug over a study period to test the efficacy and/or safety of the drug. However, the difficulty is also pronounced in a treatment setting in which DRESS may be difficult for health care providers to identify. The difficulty may further be exacerbated if multiple drugs are administered to the subject in a treatment setting or when a drug having multiple active ingredients is being tested in a clinical trial setting since it may be difficult to pinpoint that a drug or active ingredient is adverse reaction.

The systems and methods described herein may be used to detect and mitigate DRESS and other clinical events. In these examples, the event data translated into the scalable data structure 130 may include clinical trial data. For example, in operation during a clinical trial, the computer system 120 may access clinical trial data such as reported symptoms of subjects, laboratory test results, and/or other data about the subject. Some of the clinical trial data may include a symptom (such as “fever”) that was reported for a range of dates or one or more single dates. Some of the clinical trial data may include a value such as a test result that was obtained on a single date. The computer system 120 may translate the clinical data into the scalable data structure 130 as disclosed herein. The scalable data structure 130 may be used to generate the visualization 140 and/or be used for machine learning-based modeling.

For example, the computer system 120 may generate a visualization (such as the visualization 140) to show the clinical trial data translated into the scalable data structure 130. The visualization 140 may include an improved way to depict the independent clinical trial data that may occur at various ranges of dates, single dates, or multiple single dates, as well as different types of values including symptoms and quantitative test results, either of which may be binarized in the scalable data structure 130. Alternatively, or additionally, the computer system 120 may used trained models to predict whether DRESS is occurring or will occur in the subject. The visualization 140 and/or the machine learning-based modeling may improve the way in which determinations that DRESS is occurring, predictions that DRESS will occur, and/or when to discontinue one or more drugs being administered to the subject to prevent or stop DRESS.

### Forecasting Based on Machine Learning-Based Modeling of the Translated Events

DRESS and other events of interest may be identified and/or forecasted based on modeling techniques that leverage the scalable data structure 130. For example, the ML-based subsystem 128 may train and execute machine learning-based models to learn from translated events. Events represented by event markers 503 (illustrated in FIG. 5) may be used as features for training the machine learning-based models. For example, events may be selected as features for training. In these examples, the ML-based subsystem 128 may generate features for training by transforming entity data from one or more data sources 101. Selection of these features may be prone to various errors introduced by sampling, human data input errors, or other sources of error in the entity data or during feature generation. In some examples, features may be normalized based on Z-score normalization, which normalizes error in features by dividing the difference between the data and mean by standard deviation. In other examples, data may be normalized based on feature scaling, which brings all values into a range such as between 0 and 1 by dividing the difference between the data and minimum by the difference between maximum and minimum. Other normalization techniques may be used as well or instead, such as studentized residual, t-statistics, and coefficient of variation.

The ML-based subsystem 128 may use features that were optimized during a feature selection process. Feature selection refers to a process that filters in or out features to generate a filtered feature set. Examples of feature selection may include stepwise feature selection, backward elimination, forward selection, stepwise regression, lasso and ridge regression, dimensionality regression, principal component analysis, and/or other feature selection methods. The filtered feature set may include a subset of the features. Feature selection may therefore reduce the number of features used in one or more of the trained models. The feature selection process may optimize model performance. Feature selection may reduce noise and overfitting since different sensor data and different features derived from the sensor data may have different predictive impact. In some examples, the top N features may be identified by feature selection, where N is an integer. This may be used to identify the greatest signals that are most highly correlated with accurate predictions.

The ML-based subsystem 128 may generate and select features for training. For example, the ML-based subsystem 128 may access historical event data that is to serve as a training dataset. The historical event data may be associated with events that are known to have been correlated with the event of interest, such as DRESS. Thus, this historical event data may serve as a basis to train machine learning-based models to learn features that correlate with DRESS or other prediction target.

The ML-based subsystem 128 may associate the generated and selected features with a label indicating DRESS. For example, event data associated with subjects that were diagnosed with DRESS may be labeled for learning features (events such as Fever, lab results, and so forth) that preceded the diagnosis.

The ML-based subsystem 128 may train the machine learning-based models using various machine learning techniques, such as gradient boosting. Gradient boosting is a machine learning technique for regression and classification problems, which produces a prediction model in the form of an ensemble of prediction models, which may be decision trees. Gradient Boosting Machines (GBM) such as XGBoost, LightGBM, or CatBoost. may build a model in a stage-wise fashion and generalize the model by allowing optimization of an arbitrary differentiable loss function. GBM may operate on categories/sub-categories of features, making it suited for the feature sets described herein. Segmented modeling may further permit discovery of interdependencies in the data. Other machine learning techniques may be used as well, such as neural networks. A neural network, such as a recursive neural network, may refer to a computational learning system that uses a network of neurons to translate a data input of one form into a desired output. A neuron may refer to an electronic processing node implemented as a computer function, such as one or more computations. The neurons of the neural network may be arranged into layers. Each neuron of a layer may receive as input a raw value, apply a classifier weight to the raw value, and generate an output via an activation function. The activation function may include a log-sigmoid function, hyperbolic tangent, Heaviside, Gaussian, SoftMax function and/or other types of activation functions.

The machine learning techniques may employ regression and/or classification depending on particular implementations. In supervised learning, machine learning is employed to learn the mapping function from the input variable (x) (such as features) to an output variable (y) (such as a known behavior such as attrition associated with those features). The learning objective is to approximate a mapping function (f) as accurately as possible such that whenever there is a new input data (x), the output variable (y) for the dataset can be predicted. Regression techniques may generate numerical (or continuous) outputs while classification may generate categorical (or discrete) classes. Thus, regression techniques may be used for open-ended outputs while classification may be used for discrete classes (such as attrition or no attrition). Once a machine learning-based model is trained, the ML-based subsystem 128 may execute the machine learning-based model on input event data to predict or forecast an event of interest, such as DRESS.

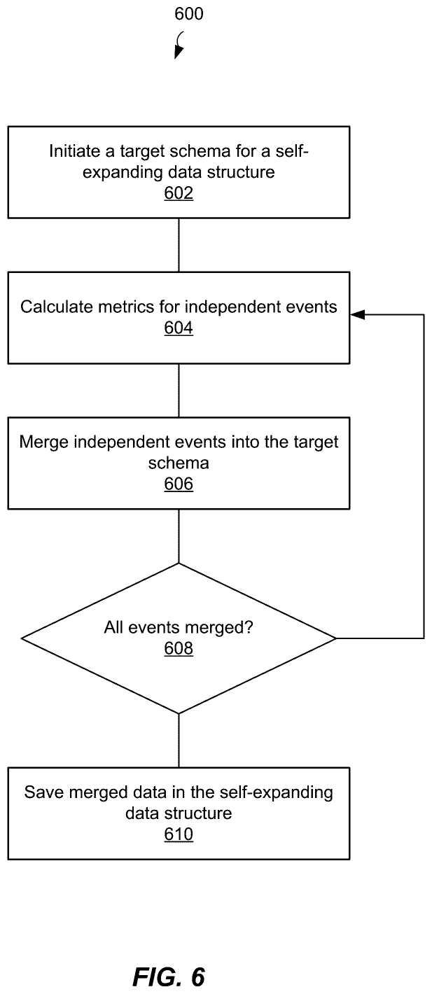

FIG. 6 illustrates an example of a method 600 of configuring and populating a scalable data structure 130 based on event data that includes independent events. Each of the independent events may be encoded as event data 103. At 602, the method 600 may include initiating a target scheme for a scalable data structure 130. Initiating the target scheme may include automatically generating a number and type of columns of the scalable data structure 130 based on values in the event data.

At 604, the method 600 may include calculate metrics for independent events.

At 606, the method 600 may include merging independent events into the target scheme.

At 608, the method 600 may include determining whether all events have been merged. If not, the method 600 may return to 604 to calculate metrics for remaining events at 604. If all events have been merged, the method 600 may, at 610, include saving the merged data in the scalable data structure 130.

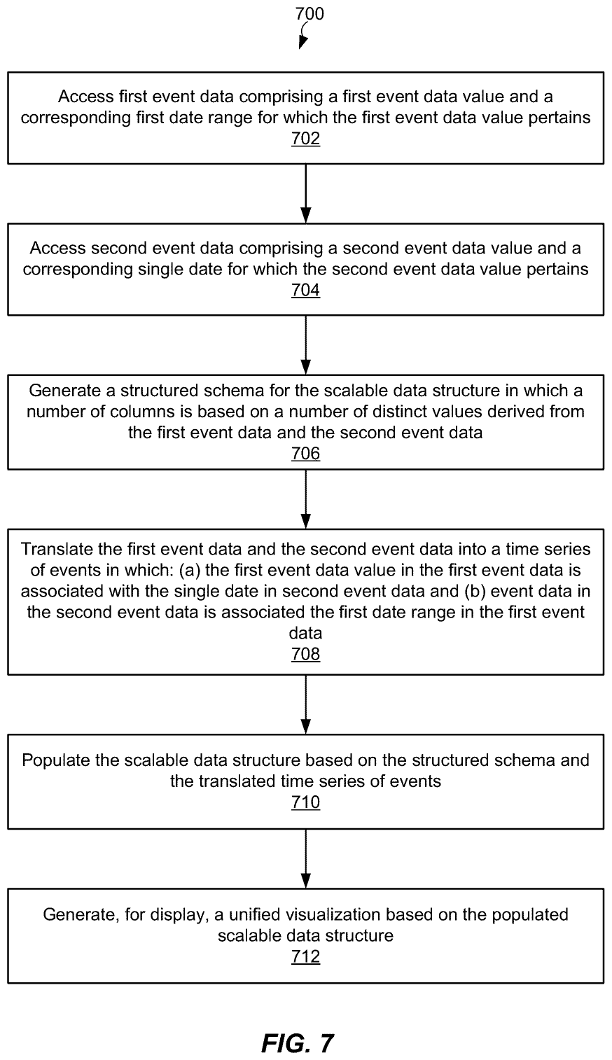

FIG. 7 illustrates an example of a method 700 of configuring, populating and using the scalable data structure 130. At 702, the method 700 may include accessing first event data (such as event data 103A-1) comprising a first event data value and a corresponding first date range for which the first event data value pertains. At 704, the method 700 may include accessing second event data (such as event data 103B-1) comprising a second event data value and a corresponding second date, from among the plurality of single dates, for which the second event data value pertains.

At 706, the method 700 may include generating a structured schema for the scalable data structure 130 in which a number of columns is based on a number of distinct values derived from the first event data and the second event data. At 708, the method 700 may include translating the first event data and the second event data into a time series of events in which: (a) the first event data value in the first event data is associated with the single date in second event data and (b) event data in the second event data is associated the first date range in the first event data.

At 710, the method 700 may include populating the scalable data structure based on the structured schema and the translated time series of events. A number of the plurality of rows is based on a start date in the first date range, an end date in the first date range, and the second single date in the second event data. An example of populating the scalable data structure is illustrated in FIGS. 3A-3C. At 712, the method 700 may include generating, for display, a visualization based on the populated scalable data structure.

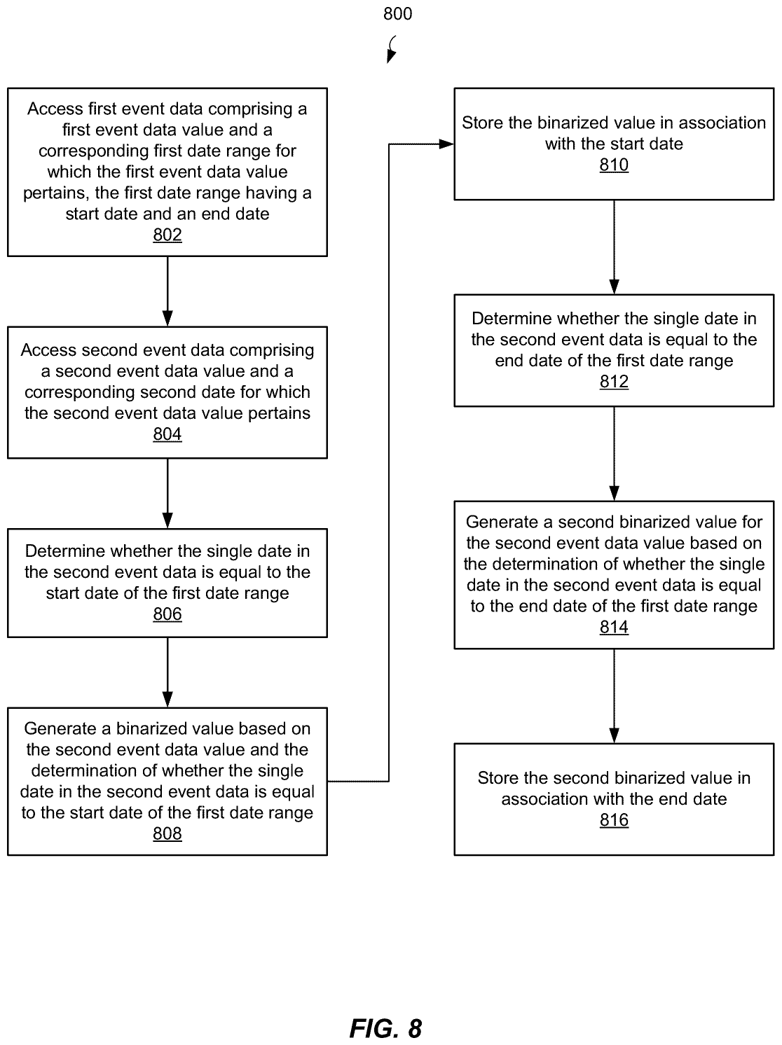

FIG. 8 illustrates an example of a method 800 of translating event data (such as event data 103). At 802, the method 800 may include accessing first event data comprising a first event data value and a corresponding first date range for which the first event data value pertains, the first date range having a start date and an end date. At 804, the method 800 may include accessing second event data comprising a second event data value and a corresponding second date for which the second event data value pertains. At 806, the method 800 may include determining whether the single date in the second event data is equal to the start date of the first date range.

At 808, the method 800 may include generating a binarized value based on the second event data value and the determination of whether the single date in the second event data is equal to the start date of the first date range. For example, if the single date in the second event data is equal to the start date of the first date range, then the second event data value will also apply to the start date of the first event data and the binarized value will reflect this. On the other hand, if the single date in the second event data is not equal to the start date of the first date range, then the second event data value will not also apply to the start date of the first event data and the binarized value will reflect this. To illustrate, referring to FIG. 2, event data 103B-1 has a single date of “1/20/22.” This single date does not equal the start date (“1/1/22”) of event data 103A-1. Thus, the binarized value will be set to “NO” or 0 to indicate that the value for the second event data 103B-1 will not apply to the start date of the first event data 103A-1. At 810, the method 800 may include storing the binarized value in association with the start date.

At 812, the method 800 may include determine whether the single date in the second event data is equal to the end date of the first date range. At 814, the method 800 may include generating a second binarized value for the second event data value based on the determination of whether the single date in the second event data is equal to the end date of the first date range. The second binarized value may be determined similar to the example described at 808. At 816, the method 800 may include storing the second binarized value in association with the end date.

A subject refers to an animal, such as a mammalian species (preferably human) or avian (e.g., bird) species, or other organism for which a physiological state such as a sleep stage may be determined. More specifically, a subject can be a vertebrate, e.g., a mammal such as a mouse, a primate, a simian or a human. Animals include farm animals, sport animals, and pets. A subject can be a healthy individual, an individual that has symptoms or signs or is suspected of having a disease (including physical or mental disease) or a predisposition to the disease, or an individual that is in need of therapy or suspected of needing therapy.

### Examples of Systems and Computing Devices

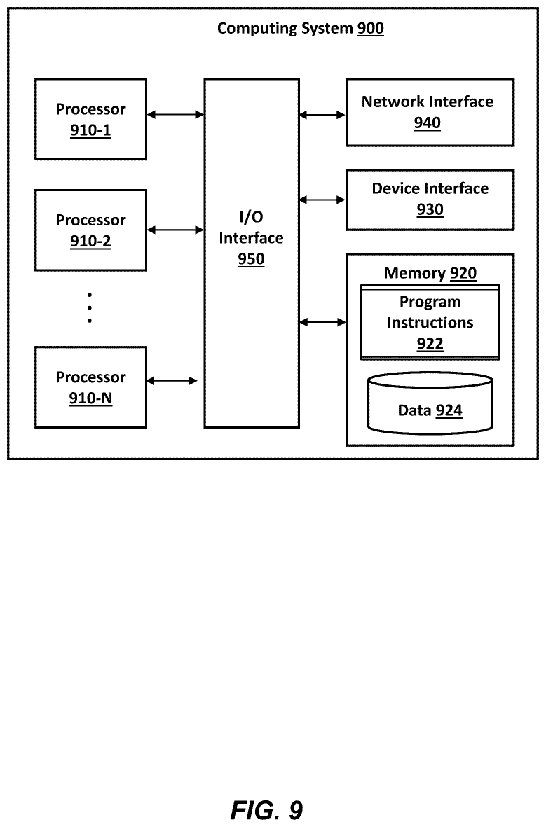

FIG. 9 illustrates an example of a computing system implemented by one or more of the features illustrated in FIG. 1. Various portions of systems and methods described herein, may include or be executed on one or more computer systems similar to computing system 900. Further, processes and modules described herein may be executed by one or more processing systems similar to that of computing system 900. In some embodiments, computer system 120, client device 160, or other components of system 100 may include some or all of the components and features of computing system 900.

Computing system 900 may include one or more processors (for example, processors 910-1N) coupled to system memory 920, an input/output I/O device interface 930, and a network interface 940 via an input/output (I/O) interface 1050. A processor may include a single processor or a plurality of processors (for example, distributed processors). A processor may be any suitable processor capable of executing or otherwise performing instructions. A processor may include a central processing unit (CPU) that carries out program instructions to perform the arithmetical, logical, and input/output operations of computing system 900. A processor may execute code (for example, processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (for example, system memory 920). Computing system 900 may be a uni-processor system including one processor (for example, processor 910-1), or a multi-processor system including any number of suitable processors (for example, 910-1-910-N). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus may also be implemented as, special purpose logic circuitry, for example, an FPGA (field programmable gate array) or an ASIC (application specific integrated circuit). Computing system 900 may include a plurality of computing devices (for example, distributed computer systems) to implement various processing functions.

I/O device interface 930 may provide an interface for connection of one or more I/O devices to computing system 900. I/O devices may include devices that receive input (for example, from a user) or output information (for example, to a user). I/O devices may include, for example, graphical user interface presented on displays (for example, a cathode ray tube (CRT) or liquid crystal display (LCD) monitor), pointing devices (for example, a computer mouse or trackball), keyboards, keypads, touchpads, scanning devices, voice recognition devices, gesture recognition devices, printers, audio speakers, microphones, cameras, or the like. I/O devices may be connected to computing system 900 through a wired or wireless connection. I/O devices may be connected to computing system 900 from a remote location. I/O devices located on remote computer system, for example, may be connected to computing system 900 via network interface 940.

Network interface 940 may include a network adapter that provides for connection of computing system 900 to a network. Network interface 940 may facilitate data exchange between computing system 900 and other devices connected to the network. Network interface 940 may support wired or wireless communication. The network may include an electronic communication network, such as the Internet, a local area network (LAN), a wide area network (WAN), a cellular communications network, or the like.

System memory 920 may store program instructions 9022 or data 9024. Program instructions 9022 may be executable by a processor (for example, one or more of processors 910-1-910-N) to implement one or more embodiments of the present techniques. Program instructions 9022 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (for example, one or more scripts stored in a markup language document), in a single file dedicated to the program in question, or in multiple coordinated files (for example, files that store one or more modules, sub programs, or portions of code). A computer program may be deployed to be executed on one or more computer processors located locally at one site or distributed across multiple remote sites and interconnected by a communication network.

System memory 920 may include a tangible program carrier having program instructions stored thereon. A tangible program carrier may include a non-transitory computer readable storage medium. A non-transitory computer readable storage medium may include a machine-readable storage device, a machine-readable storage substrate, a memory device, or any combination thereof. Non-transitory computer readable storage medium may include non-volatile memory (for example, flash memory, ROM, PROM, EPROM, EEPROM memory), volatile memory (for example, random access memory (RAM), static random access memory (SRAM), synchronous dynamic RAM (SDRAM)), bulk storage memory (for example, CD-ROM and/or DVD-ROM, hard-drives), or the like. System memory 920 may include a non-transitory computer readable storage medium that may have program instructions stored thereon that are executable by a computer processor (for example, one or more of processors 910-1-910-N) to cause the subject matter and the functional operations described herein. A memory (for example, system memory 920) may include a single memory device and/or a plurality of memory devices (for example, distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 950 may coordinate I/O traffic between processors 910-1-910-N, system memory 920, network interface 940, I/O devices, and/or other peripheral devices. I/O interface 1050 may perform protocol, timing, or other data transformations to convert data signals from one component (for example, system memory 920) into a format suitable for use by another component (for example, processor 910-1, processor 910-2, . . . , processor 910-N). I/O interface 950 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computing system 900 or multiple computing systems 900 configured to host different portions or instances of embodiments. Multiple computing systems 900 may provide for parallel or sequential processing/execution of one or more portions of the techniques described herein.

Those skilled in the art will appreciate that computing system 900 is merely illustrative and is not intended to limit the scope of the techniques described herein. Computing system 900 may include any combination of devices or software that may perform or otherwise provide for the performance of the techniques described herein. For example, computing system 900 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computing system 900 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (for example, as instructions or structured data) on a computer-accessible medium or a portable article to be read by an appropriate drive, various examples of which are described above. In some embodiments, instructions stored on a computer-accessible medium separate from computing system 900 may be transmitted to computing system 900 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

At 1002, the method 1000 may include administering a pharmaceutical to a subject. The subject may be administered with the pharmaceutical for various reasons such as to treat or manage a medical condition, test efficacy and safety of the pharmaceutical during a clinical trial, and/or other reasons. The pharmaceutical may include medications such as non-steroidal anti-inflammatory drugs, captopril, mood stabilizers, antiretrovirals and/or other medications. In some examples, the pharmaceutical can include vaccines, supplements, and/or other substances that can be administered to the subject.

At 1004, the method 1000 may include obtaining data from a scalable data structure (such as scalable data structure 130) with first event data (such as event data 103A-1) and second event data (such as event data 103B-1). The first event data may indicate a first event associated with the subject during a first date range after the administering and the second event data indicating a second event experienced by the subject at a single date after the administering. A number of columns of the scalable data structure may be based on a number of distinct values derived from the first event data and the second event data and a number of a plurality of rows may be based on a start date in the first date range, an end date in the first date range, and the second single date in the second event data and wherein each row from among the plurality of rows has a plurality of columns each corresponding to the distinct values derived from the first event data and the second event data.

At 1006, the method 1000 may include determining that the subject had or is having an adverse reaction to the pharmaceutical based on the data obtained from the scalable data structure. The example may include DRESS and/or other adverse reaction. At 1008, the method 1000 may include discontinuing administration of the pharmaceutical based on the determination.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (for example, content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to cost constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (in other words, meaning having the potential to), rather than the mandatory sense (in other words, meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, in other words, encompassing both “and” and “or.” Terms describing conditional relationships, for example, “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, for example, “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, for example, the antecedent is relevant to the likelihood of the consequent occurring. Statements in which a plurality of attributes or functions are mapped to a plurality of objects (for example, one or more processors performing steps A, B, C, and D) encompasses both all such attributes or functions being mapped to all such objects and subsets of the attributes or functions being mapped to subsets of the attributes or functions (for example, both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B may include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, in other words, each does not necessarily mean each and every. Limitations as to sequence of recited steps should not be read into the claims unless explicitly specified, for example, with explicit language like “after performing X, performing Y,” in contrast to statements that might be improperly argued to imply sequence limitations, like “performing X on items, performing Y on the X′ed items,” used for purposes of making claims more readable rather than specifying sequence. Statements referring to “at least Z of A, B, and C,” and the like (for example, “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, for example, reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, for example, text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and may be implemented in the form of data that causes functionality to be invoked, for example, in the form of arguments of a function or API call. To the extent bespoke noun phrases are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (for example, articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

This written description uses examples to disclose the embodiments, including the best mode, and to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system, comprising:
a processor programmed to:
access first event data comprising a first event data value and a corresponding first date range for which the first event data value pertains;
access second event data comprising a second event data value and a corresponding single date for which the second event data value pertains;
generate a structured schema for a scalable data structure in which a number of columns is based on a number of distinct values derived from the first event data and the second event data;
translate the first event data and the second event data into a time series of events in which: (a) the first event data value in the first event data is associated with the single date in second event data and (b) the second event data value in the second event data is associated the first date range in the first event data;
populate the scalable data structure based on the structured schema and the translated time series of events,
wherein a number of a plurality of rows is based on a start date in the first date range, an end date in the first date range, and the second single date in the second event data and wherein each row from among the plurality of rows has a plurality of columns each corresponding to the distinct values derived from the first event data and the second event data; and
generate, for display, a visualization based on the populated scalable data structure.

2. The system of claim 1, wherein to generate the structured schema, the processor is further programmed to:
parse the first event data value from the first event data; and
generate a first column in the structured schema, the first column having a first column name based on the first event data value.

3. The system of claim 2, wherein to generate the structured schema, the processor is further programmed to:
parse the second event data value from the second event data; and
generate a second column in the structured schema, the second column having a second column name based on the second event data value.

4. The system of claim 1, wherein to translate the first event data, the processor is further programmed to:
determine whether the single date in the second event data is equal to the start date of the first date range;
generate a binarized value based on the second event data value and the determination of whether the single date in the second event data is equal to the start date of the first date range;
store the binarized value as a column value of a column for a row corresponding to the start date.

5. The system of claim 4, wherein to translate the second event data, the processor is further programmed to:
determine whether the single date in the second event data is equal to the end date of the first date range;
generate a second binarized value based on the second event data value and the determination of whether the single date in the second event data is equal to the end date of the first date range;
store the second binarized value as a second column value of a second column for a second row corresponding to the end date.

6. The system of claim 1, wherein to translate the second event data, the processor is further programmed to:
determine whether the single date in the second event data is within the first date range;
generate a binarized value based on the first event data value and the determination of whether the single date in the second event data is within the first date range;
store the binarized value as a column value of a column for a row corresponding to the single date.

7. The system of claim 1, wherein the first event data value pertains to a symptom that was reported during the first date range.

8. The system of claim 1, wherein the second event data value pertains to a test result that was obtained at the single date.

9. The system of claim 1, wherein to generate the visualization, the processor is further programmed to:
generate a timeline based on rows in the scalable data structure;
for each row in the scalable data structure:
for each column in the scalable data structure, determine whether a column value for the column represents an event of interest and generate an event marker along the timeline corresponding to the row depending on whether the column value for the column represents an event of interest.

10. A method, comprising:
accessing, by a processor, first event data comprising a first event data value and a corresponding first date range for which the first event data value pertains;
accessing, by the processor, second event data comprising a second event data value and a corresponding single date for which the second event data value pertains;
generating, by the processor, a structured schema for a scalable data structure in which a number of columns is based on a number of distinct values derived from the first event data and the second event data;
translating, by the processor, the first event data and the second event data into a time series of events in which: (a) the first event data value in the first event data is associated with the second date in second event data and (b) the second event data value in the second event data is associated the first date range in the first event data;
populating, by the processor, the scalable data structure based on the structured schema and the translated time series of events,
wherein a number of a plurality of rows is based on a start date in the first date range, an end date in the first date range, and the second single date in the second event data and wherein each row from among the plurality of rows has a plurality of columns each corresponding to the distinct values derived from the first event data and the second event data; and
generating, by the processor, for display, a visualization based on the populated scalable data structure.

11. The method of claim 10, wherein generating the structured schema comprises:
parsing the first event data value from the first event data; and
generating a first column in the structured schema, the first column having a first column name based on the first event data value.

12. The method of claim 11, wherein generating the structured schema comprises:
parsing the second event data value from the second event data; and
generating a second column in the structured schema, the second column having a second column name based on the second event data value.

13. The method of claim 10, wherein translating the first event data comprises:
determining whether the single date in the second event data is equal to the start date of the first date range;
generating a binarized value based on the second event data value and the determination of whether the single date in the second event data is equal to the start date of the first date range; and
storing the binarized value as a column value of a column for a row corresponding to the start date.

14. The method of claim 13, wherein translating the second event data comprises:
determining whether the single date in the second event data is equal to the end date of the first date range;
generating a second binarized value based on the second event data value and the determination of whether the single date in the second event data is equal to the end date of the first date range; and
storing the second binarized value as a second column value of a second column for a second row corresponding to the end date.

15. The method of claim 10, wherein translating the second event data comprises:
determining whether the single date in the second event data is within the first date range;
generating a binarized value based on the first event data value and the determination of whether the single date in the second event data is within the first date range; and
storing the binarized value as a column value of a column for a row corresponding to the single date.

16. The method of claim 10, wherein the first event data value pertains to a symptom that was reported during the first date range.

17. The method of claim 10, wherein the second event data value pertains to a test result that was obtained at the single date.

18. The method of claim 10, the method further comprising:
generating a timeline based on rows in the scalable data structure;
for each row in the scalable data structure:
for each column in the scalable data structure, determining whether a column value for the column represents an event of interest and generating an event marker along the timeline corresponding to the row depending on whether the column value for the column represents an event of interest.

19. A non-transitory computer readable medium storing instructions that, when executed by a processor, programs the processor to:
access first event data comprising a first event data value and a corresponding first date range for which the first event data value pertains, the first date range having a start date and an end date;
access second event data comprising a second event data value and a corresponding single date for which the second event data value pertains;
determine whether the single date in the second event data is equal to the start date of the first date range;
generate a binarized value based on the second event data value and the determination of whether the single date in the second event data is equal to the start date of the first date range;
store the binarized value in association with the start date;
determine whether the single date in the second event data is equal to the end date of the first date range;
generate a second binarized value for the second event data value based on the determination of whether the single date in the second event data is equal to the end date of the first date range; and
store the second binarized value in association with the end date.

20. The non-transitory computer readable medium storing instructions of claim 19, wherein the instructions, when executed, further programs the processor to:
determine whether the single date in the second event data is within the first date range;
generate a third binarized value based on the first event data value and the determination of whether the single date in the second event data is within the first date range;
store the third binarized value in association with the single date.

21. A method of administering a treatment, comprising:
administering a pharmaceutical to a subject;
obtaining data from a scalable data structure with first event data and second event data, the first event data indicating a first event associated with the subject during a first date range after the administering and the second event data indicating a second event experienced by the subject at a single date after the administering,
wherein a number of columns of the scalable data structure is based on a number of distinct values derived from the first event data and the second event data and a number of a plurality of rows is based on a start date in the first date range, an end date in the first date range, and the second single date in the second event data and wherein each row from among the plurality of rows has a plurality of columns each corresponding to the distinct values derived from the first event data and the second event data;
determining that the subject had or is having an adverse reaction to the pharmaceutical based on the data obtained from the scalable data structure; and
discontinuing administration of the pharmaceutical based on the determination.

22. The method of claim 21, further comprising:
generating a visualization based on the first event data and the second event data; and
transmitting the visualization to support clinical diagnostics in a medical decision support system.

23. The method of claim 21, wherein the adverse condition is Drug Reaction with Eosinophilia and Systemic Symptoms.
